(19) United States
(12) Patent Application Publication
(10) Pub. No.: US 2022/0226282 A1
(43) Pub. Date: Jul. 21, 2022

(54) Natural Product Antidotes against Botulinum Neurotoxins

(71) Applicant: Prime Bio, Inc., Dartmouth, MA (US)

(72) Inventors: Bal Ram Singh, Dartmouth, MA (US); Kruti Patel, Dartmouth, MA (US)

(21) Appl. No.: 17/151,233

(22) Filed: Jan. 18, 2021

(51) Int. Cl. A61K 31/366; A61P 31/04; A61K 47/24; A61K 47/26; A61K 9/50; A61K 9/127; A61K 9/51

(52) U.S. Cl. CPC A61K 31/366; A61P 31/04; A61K 47/24; A61K 9/5123; A61K 9/5015; A61K 9/127; A61K 47/26

(57) ABSTRACT

The present invention relates to a method of treating an individual suffering from botulism comprising administering to the individual a composition comprising a therapeutically effective amount of Compound 1 or its pharmaceutically acceptable salts or derivatives thereof.

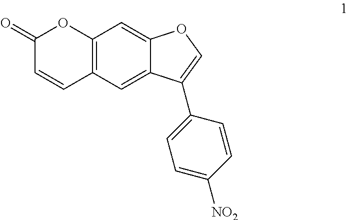 [SMILES: O=c1ccc2cc3c(-c4ccc([N+](=O)[O-])cc4)coc3cc2o1]

### FIELD OF THE INVENTION

The present invention relates to a method of treating an individual suffering from botulism comprising administering to the individual a composition comprising a therapeutically effective amount of Compound 1 or its pharmaceutically acceptable salts or derivatives thereof.

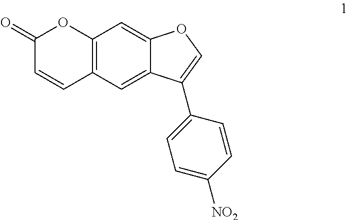 [SMILES: O=c1ccc2cc3c(-c4ccc([N+](=O)[O-])cc4)coc3cc2o1]

Chemically, Compound 1 is 3-(4-nitrophenyl)-7H-furo[3,2-g]chromen-7-one, which blocks the biological relevant enzyme activity of botulinum neurotoxin, thus, act as a natural antidote against botulinum neurotoxins.

### BACKGROUND OF THE INVENTION

Botulinum Neurotoxins (BoNTs) are proteins responsible for the deadly paralytic disease called botulism. Extreme toxicity, ease of production, and lack of antidotes against BoNT makes it a “Category A” biothreat agent, according to the United States Center of Disease Control and Prevention. These deadly toxins, in minute quantities, estimated human LD₅₀ (i.v.) of 0.1 ng/kg body weight, cause fatal flaccid paralysis by blocking neurotransmitter release.

BoNT is designated as a “Category A” agent on the National Institute of Allergy and Infectious Diseases (NIAID) priority-pathogen list and poses a significant threat to public health. Due to their high toxicity and relatively easy production, BoNTs create maximum fear among populations concerned with bioterror agents. Contamination of restaurant, catered, or commercial foodstuffs, or beverages could cause illness in a large number of consumers. Aerosol exposure of BoNTs does not occur naturally, but could be attempted by bioterrorists to achieve widespread effect. A single gram of crystalline toxin, evenly dispersed and inhaled, would kill more than one million people, although technical factors would make such dissemination difficult. A more realistic scenario suggests that less than one gram of BoNT, if distributed into a food supply, such as milk, could cause more than 100,000 casualties. Currently, there is no effective antidote available, except the equine antitoxin sera, and no safe prophylaxis against botulism. There is an urgent need to develop both prophylactic and therapeutic agents against BoNTs. The challenge of developing a more effective treatment for botulism has been recognized by NIAID, and has been among NIAID's highest priority.

BoNTs are produced by the bacteria C. botulinum and are released into the medium after bacterial lysis as an inactive 150 kDa single polypeptide chain. Seven serotypes of the botulinum neurotoxins are botulinum neurotoxin A, B, C, D, E, F, and G. The botulinum neurotoxin is 150 kDa, and the toxin produced in the bacteria is in the form of a complex, containing the neurotoxin and neurotoxin associated proteins (NAPs). The 150 kDa protein is post-translationally proteolyzed (nicked) by bacterial proteases to form the biologically active di-chain neurotoxin, composed of a 100 kDa heavy chain (HC) and a 50 kDa light chain (LC), linked through a disulfide bond and non-covalent protein interactions. All seven serotypes have a similar mechanism of action facilitated by three common protein domains with specific functions, which work together to establish toxicity. An active toxin consists of: i) a neuron-specific receptor binding domain-50 kDa carboxy-terminal heavy chain (HCC), ii) a membrane translocation domain-50 kDa amino-terminal heavy chain (HCN), and iii) a catalytic domain-50 kDa zinc endo-peptidase light chain (LC). A single disulfide bond bridges light chain with the amino terminal heavy chain.

Naturally botulism can be caused by three ways: (i) foodborne botulism caused by ingestion of toxin from foods; (ii) through a wound caused by contamination of a wound by BoNT producing spores/bacteria; and (iii) infant botulism caused by colonization of the digestive tract by the bacterium in children.

Among seven serotypes of botulinum neurotoxins types A, B, E, and rare cases, F cause botulism in humans. Types C and D cause disease in birds and mammals. Type G, identified in 1970, has not yet been confirmed as a cause of illness in humans or animals.

Among all seven serotypes of botulinum neurotoxins, BoNT/A is the most potent, and it takes more than six months to recover from botulism caused by BoNT/A. The only available therapy for BoNT is an equine antitoxin antibody or/and a protracted respiratory support system. Even the antibody treatment can only prevent further exposure of the toxin and cannot treat the already intoxicated neurons.

The long-lasting endopeptidase activity of the BoNTs is a critical biological activity inside the nerve cell. It catalyzes proteolysis of the SNARE proteins involved in the exocytosis of acetylcholine, thus causing muscle paralysis. Therefore, there is an urgent need to identify and develop oral candidates that can inhibit BoNT's endopeptidase activity and act as ultimate therapeutics for treating botulism. Further, a small drug-like molecule could turn into the most effective drug. Once developed into drugs, these have the advantage of higher stability and membrane permeability to reach the target, in the case of nerve cells poisoned by BoNTs.

Small molecule inhibitors of BoNTs provide a better alternative due to low toxicity, no reported immunogenicity, high specificity, high effectiveness, long shelf-life, and low cost of synthesis. Small molecules derived from natural sources such as plants provide extra safety and least side effects due to their evolutionary traits and compatibility with human physiology.

The present invention employs the use of a derivative of a natural compound produced by plants. Psoralen is produced by plants. Ficus carica (fig) is the most abundant source of psoralens. They are also found in small quantities in Ammi visnaga (bisnaga), Pastinaca sativa (parsnip), Petroselinum crispum (parsley), Levisticum officinale (lovage), Foeniculum vulgare (fruit, i.e., fennel seeds), Daucus carota (carrot), Psoralea corylifolia (babchi), and Apium graveolens (celery). Compound 1 is 3-(4-nitrophenyl)-7H-furo[3,2-g]chromen-7-one, a nitrophenyl analog of psoralen.

### BRIEF DESCRIPTION OF THE DRAWINGS

FIG. 1 shows IC₅₀ graph of Compound 1, showing % inhibition of the LCA endopeptidase activity vs. concentration of Compound 1.

FIG. 2 shows a double-reciprocal plot of substrate concentration versus velocity.

FIG. 3 (a)-(c) depicts the effect of Compound 1 on the intrinsic fluorescence of BoNT/A LC with an excitation wavelength of 295 nm.
FIG. 3a depicts the fluorescence intensity at emission maxima of BoNT/A LC (204) corresponding concentration-dependent addition of Compound 1 (20 μM, 50 μM, 100 μM and 200 μM) in the ratio of (1:10, 1:25, 1:50, 1:100).
FIG. 3b shows the correction of the inner filter effect. Percentage ratio fluorescence signal reduction of F-observed was compared with F-correction when incubated with Compound 1 in the concentration manner.
FIG. 3c depicts the Stern-Volmer plot of the fluorescence intensities in the absence and presence of Compound 1 quencher (F₀/F) versus Compound 1 concentration in M at 298.15 K and 310.15 K temperature.

FIG. 4 (a)-(b) represents Isothermal Titration calorimetry (ITC) of Compound 1 with the BoNT/A LC interactions.
FIG. 4a: Raw data obtained for 25 injections of 10 μl of 0.4 mM Compound 1 solution into the sample cell containing 40 μM BoNT/A LC (after subtraction of the integration baseline.)
FIG. 4b: Normalized integrated enthalpies plotted against the molar ratio of Compound 1 to BoNT/A LC.

### SUMMARY OF THE INVENTION

The present invention relates to a method of treating an individual suffering from botulism comprising: administering to the individual a composition comprising a therapeutically effective amount of Compound 1 or its pharmaceutically acceptable salts or derivatives thereof.

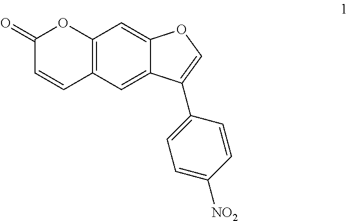 [SMILES: O=c1ccc2cc3c(-c4ccc([N+](=O)[O-])cc4)coc3cc2o1]

Chemically, Compound 1 is 3-(4-nitrophenyl)-7H-furo[3,2-g]chromen-7-one, which is a nitrophenyl psoralen (NPP).

The present invention relates to a composition comprising a therapeutically effective amount of 3-(4-nitrophenyl)-7H-furo[3,2-g]chromen-7-one or its pharmaceutically acceptable salts and one or more excipients.

The present invention relates to a method of treating an individual suffering from botulism comprising: administering to the individual a therapeutically effective amount of Compound 1 or its pharmaceutically acceptable salts or derivatives thereof in combination with other drugs.

Compound 1 is also suitable for oral delivery. It obviates the need for an antibody injection or artificial ventilation for the treatment of botulism. Thus, it solves the problem of storing biological molecules like antibodies requiring refrigeration and safety concerns related to the injectable delivery such as bolus, fast-acting, rapid release of the drug. Further, Compound 1 provides a better alternative due to low toxicity, no reported immunogenicity, high specificity, high effectiveness, long shelf-life, and low cost of synthesis.

### DETAILED DESCRIPTION OF THE INVENTION

As used herein, the word “a” or “plurality” before a noun represents one or more of the particular nouns. For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this disclosure belongs. Methods and materials are described herein for use in the present disclosure; other suitable methods and materials known in the art can also be used. The materials, methods, and examples are illustrative only and are not intended to be limiting. All publications, patent applications, patents, sequences, database entries, and other references mentioned herein are incorporated by reference in their entirety. In case of conflict, the present specification, including definitions, will control.

The present invention relates to a method of treating an individual suffering from botulism comprising: administering to the individual a pharmaceutically acceptable composition comprising a therapeutically effective amount of Compound 1 or its pharmaceutically acceptable salts or derivatives thereof.

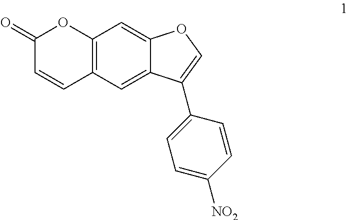 [SMILES: O=c1ccc2cc3c(-c4ccc([N+](=O)[O-])cc4)coc3cc2o1]

Chemically, Compound 1 is 3-(4-nitrophenyl)-7H-furo[3,2-g]chromen-7-one, which is a nitrophenyl psoralen (NPP). Psoralen is a natural product produced by plants. Ficus carica (fig) is the most abundant source of psoralens. They are also found in small quantities in Ammi visnaga (bisnaga), Pastinaca sativa (parsnip), Petroselinum crispum (parsley), Levisticum officinale (lovage), Foeniculum vulgare (fruit, i.e., fennel seeds), Daucus carota (carrot), Psoralea corylifolia (babchi), and Apium graveolens (celery).

The term ‘pharmaceutically acceptable salts’ means salts prepared by alkali metal and alkaline earth metal hydroxides or carbonates or bicarbonates or with any organic amines.

The term ‘derivatives’ means, in compound 1 the nitro group may be replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc. The lactum may be opened to form a carboxylic acid group or converted to ester amide, aldehyde. Further, the rings may be substituted with amine, alkylated amines, acyl groups, substituted or unsubstituted alkyl groups with maximum of four carbons, hydroxyl group, halogen, aldehyde, carboxylic acid, ester, amide, substituted amide etc. The carboxylic acid group obtained by opening lactum may be converted to corresponding salts prepared by alkali metal and alkaline earth metal hydroxides or carbonates or bicarbonates or with any organic amines.

The term ‘pharmaceutically acceptable composition’ means a composition that is physiologically tolerable and do not typically produce an allergic or similar untoward reaction when administered to an individual, preferably a human subject.

The pharmaceutically acceptable composition further comprises one or more pharmaceutically acceptable excipients.

The “pharmaceutically acceptable excipients” is selected from one or more of diluents, carriers or fillers, binders, disintegrants, lubricants, suspending agents, solubilizing agents/surfactants, stabilizing agents, glidants, antioxidants, colors, flavors, preservatives, or mixtures thereof.

Suitable diluents include ethanol, glycerol, dimethyl sulfoxide (DMSO), water, or a mixture thereof.

Suitable carriers or fillers include one or more sugars, such as dextrose, glucose, lactose; sugar alcohols, such as sorbitol, xylitol, mannitol; cellulose derivatives, such as powdered cellulose, microcrystalline cellulose; starches, such as corn starch, pregelatinized starch, maize starch; or mixtures thereof.

Suitable binders include one or more of starch; gelatin; polyethylene glycol; cellulose derivatives, such as hydroxypropylmethyl cellulose, hydroxypropyl cellulose, methylcellulose, carboxymethyl cellulose; natural and synthetic gums, such as xanthan gum, gum acacia, tragacanth; water-soluble vinylpyrrolidone polymers, such as polyvinylpyrrolidone, copolymer of vinylpyrrolidone and vinyl acetate; natural sugars such as glucose, anhydrous lactose, free-flow lactose, beta-lactose; sugars alcohols, such as sorbitol, mannitol; corn sweeteners; or mixtures thereof.

Suitable disintegrants include sodium starch glycolate, croscarmellose sodium, crospovidone, cornstarch, or mixtures thereof.

Suitable lubricants include sodium oleate, sodium stearate, magnesium stearate, sodium benzoate, sodium acetate, sodium chloride, or mixtures thereof.

Suitable solubilizing agents/surfactants include one or more of sodium lauryl sulphate, polyethylene sorbitol esters such as Tween 80, or mixtures thereof.

Suitable glidants include one or more of magnesium stearate, talc, sodium stearyl fumarate, colloidal silicon dioxide, and mixtures thereof.

Suitable antioxidants include but not limited to alkyl gallates (e.g. dodecyl-, ethyl-, octyl-, propyl-gallate), butylated hydroxyanisole, butylated hydroxytoluene, tocopherols (e.g. alpha tocopherol), ascorbic acid palmitate, ascorbic acid, sodium ascorbate, potassium and sodium salts of sulphurous acid (e.g. bisulphites, metabisulphites, sulphites), flavonoides (rutin, quercetin, caffeic acid), or mixtures thereof.

Suitable colors, flavors, and preservatives include FDA approved, safe, and edible food colors, flavors, and preservatives.

The “pharmaceutically acceptable excipients” is selected from one or more of aspartame, phenylalanine, benzalkonium chloride, benzoic acid and benzoates, benzyl alcohol, boric acid and borate, cyclodextrins, dextrans, ethanol, fructose, sorbitol, lactose, phosphates, polysorbates, proline, propylene glycol and esters, sodium lauryl sulfates, wheat starch (containing gluten), gelatin, cellulose, cellulose derivatives, polyvinyl pyrrolidone, and starch.

Botulism is caused by botulinum neurotoxin produced by clostridium botulinum. Compound 1 blocks the biological relevant enzyme activity of botulinum neurotoxin, thus, acting as a natural antidote against botulinum neurotoxins. The botulinum neurotoxin is type A, B, C, D, E, F, G, X, more preferably botulinum neurotoxin types A, B, E and F.

According to one embodiment of the invention, Compound 1 is administered by oral administration, nasal administration, topical administration, parenteral administration, rectal administration, systemic administration, intramuscular administration, or intravenous administration, more preferably, by oral administration or nasal administration.

According to other embodiment of the invention, the therapeutically effective amount of Compound 1 is in the range of <1 to 10 μM /Kg.

According to another embodiment of the invention, the composition further comprises lipids and/or Compound 1 is encapsulated in microspheres, liposomes, or nanoparticles linked to detoxified recombinant BoNT.

The term “lipid” refers to a group of organic compounds that include, but are not limited to, esters of fatty acids and are characterized by being insoluble in water but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids. The lipids employed in the invention are neutral lipids, non-cationic lipids, anionic lipids, cationic lipids, hydrophobic lipids, herbal lipids, oily solutions, or mixture thereof.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethano lamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “anionic lipid” refers to any lipid that is negatively charged at physiological pH. These lipids include but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines (glutaryl PE), lysylphosphatidylglycerols, palmitoyloleyolphosphatidylglycerol (POPG), and other anionic modifying groups joined to neutral lipids.

The term “cationic lipid” refers to any of a number of lipid species that carry a net positive charge at a selected pH, such as physiological pH (e.g., pH of about 7.0). It has been surprisingly found that cationic lipids comprising alkyl chains with multiple sites of unsaturation, e.g., at least two or three sites of unsaturation, are particularly useful for forming lipid particles with increased membrane fluidity. Non-limiting examples of cationic lipids are described in detail herein. In some cases, the cationic lipids comprise a protonatable tertiary amine (e.g., pH titratable) head group, Cis alkyl chains, ether linkages between the head group and alkyl chains, and 0 to 3 double bonds. Such lipids include, e.g., DSDMA, DLinDMA, DLenDMA, and DODMA.

The term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N-N-dialkylamino, 1,2-diacyloxy-3-aminopropane, and 1,2-dialkyl-3-aminopropane.

The term “herbal lipids” refers to lipids derived from plant sources.

The lipids may be composed of oily solutions selected from the group consisting of triglyceride, ethyl icosapentate, tocopherol nicotinate, teprenone, indomethacin franesil, and dronabinol.

The term “encapsulation” refers to entrapping active agents into a biodegradable and biocompatible polymeric matrix or shell. Compound 1 can be encapsulated in microspheres, liposomes, or nanoparticles. “Microspheres” are small spherical particles, with a diameter 1 μm to 1000 μm. Microspheres are of two types microcapsules and micromatrices. Microcapsules are those in which entrapped active agent is distinctly surrounded by distinct capsule wall. Micromatrices are those in which entrapped active agent is dispersed throughout the matrix. “Liposomes” are simple microscopic, concentric bilayered vesicles in which an aqueous volume is entirely enclosed by a membranous lipid bilayer. Water-soluble active agents are entrapped in the aqueous volume, and lipid-soluble drugs are entrapped within the bilayer. “Nanoparticles” are extremely small particles, with a diameter of 1 nm to 1000 nm. Like microspheres, nanoparticles are also of two types nanocapsules and nanomatrices.

### Protein (BoNT)—Inhibitor (Compound) Binding Studies

More than 1024 compounds were tested in three batches, out of which 41 selected inhibitors were chosen from the initial screening based on their effectiveness.

The inhibitory activity of these compounds was tested using a truncated form of a membrane protein SNAP-25 and a 13 amino acid long fluorescence resonance energy transfer (FRET) based peptide was used as the substrate to assay BoNT/A LC endopeptidase activity. β-alanine was added to the C-terminus for the efficient labeling of the fluorophore (Fluorescein-5-isothiocyanate, FITC). The sequence of the peptide is FITC-b (Ala)-Thr-(D-Arg)-Ile-Asp-Gln-Ala-Asn-Gln-Arg-Ala-Thr-Lys (DABCYL)-Norleucine-CONH₂. The 4-dimethylaminoazobenzene-4′-carboxylic acid (DABCYL) component served as the FITC quencher. The catalytic domain of BoNT/A cleaves the peptide cleavage site is between its Gln and Arg residues. The FRET substrate peptide was synthesized by New England Peptide (Gardener, Mass.) and possessed a purity of greater than 95%.

The peptide substrate stock solution was prepared using distilled water to obtain a 10 μM solution. BoNT/A LC is diluted with assay buffer to prepare a 100 nM stock solution. To screen for an effective inhibitor, 50 μL of BoNT/A LC is transferred into 96-well clear bottom microtiter plates (Corning, Corning, N.Y.) and compounds (5 mg/ml in DMSO) were transferred into each well. The final compound concentration is 25 μg/ml in each well. These compounds and BoNT/A LC were pre-incubated at 37° C. for 30 min. 50 μL peptide substrate is added to the reaction mixture of enzyme and inhibitor. Each plate contained at least three wells for positive controls and three wells for negative controls. The plates were incubated at 37° C. for 30 minutes to allow the endopeptidase reaction to occur. The positive control was BoNT/A LC without inhibitor but with the DMSO. The negative control was assay buffer without either BoNT/A LC or inhibitor. The plates were read using a SpectraMax M5 fluorescence microplate reader (Molecular Devices). The excitation wavelength used was 490 nm with an emission wavelength of 523 nm with auto cutoff.

The IC₅₀ value for these compounds using FRET peptide substrate-based endopeptidase assay and dose-dependent inhibition curve was established. Using 50 nM LC of BoNT/A and different concentrations of the inhibitor incubated at 37° C. for 30 min before adding the 5 μM of >95% purified substrate peptide. Enzyme and substrate mixture was incubated at 37° C. for 3 to 4 hours while reading plate every 30 min using excitation wavelength of 490 nm and emission wavelength of 523 nm. The IC₅₀ value was interpolated from the concentration-response curve using a non-linear polynomial regression.

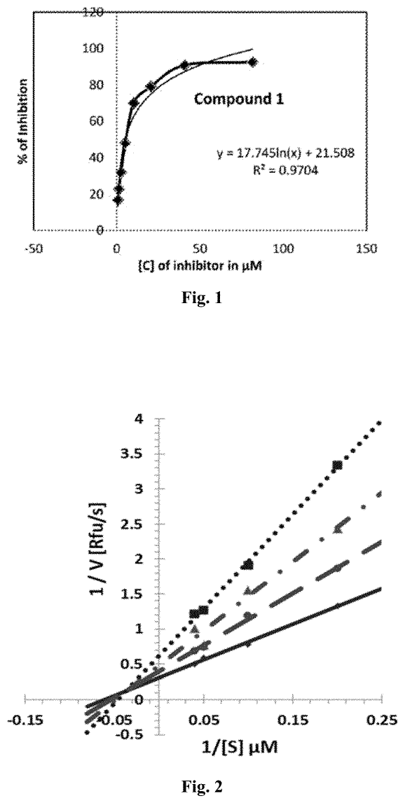

The compounds exhibited a dose-dependent inhibition effect against the endopeptidase activity of rLCA. The IC₅₀ value of these compounds is in the low micro molar range. 10 different concentrations of inhibitor were used. Plots of [concentration of inhibitor] vs. % inhibition shown clear saturation points for Compound 1 reached near 100% inhibition about 81 μM (FIG. 1). Since the enzyme concentration we used in the inhibition assay is 50 nM, which means that 50% LCA inhibition is achieved when the molar ratio (inhibitor: rLCA) is 95:1 for the Compound 1. The potency of the Compound 1 has been tested using biochemical assays, cellular assay, and in mouse phrenic nerve-hemidiaphragm preparations. It has been shown to be an efficient antidote with the ability to mitigate the paralytic actions of BoNT.

Further, testing of these compounds with more elaborate testing resulted that IASMLN4493 (NPP), i.e., Compound 1 was the one with high effectiveness against BoNT/A endopeptidase.

### Enzyme Kinetic Studies

The enzyme kinetics was carried out using the 13-mer peptide-based substrate. A series of concentrations from 5 to 25 μM of substrate was used (e.g., 5, 10, 20, and 25 μM) for enzyme kinetic study, with 50 nM of LC of BoNT/A. The reaction buffer was same as the HTS assay described above. The reactions were carried out at 37° C., with the monitoring of fluorescence in the first 10 min to calculate the initial velocity of the reaction. The fluorescence was within the linear range for the contractions of substrate chosen above. To evaluate the inhibition kinetics, Compound 1 was pre-incubated with the LC of BoNT/A at 37° C. for 30 min before adding the substrate. The concentrations of Compound 1 used were chosen near its IC₅₀ values. All the results are the average of triplicate measurements.

The enzyme kinetic studies were carried out on Compound 1. The Lineweaver-Burk plots (FIG. 2) were constructed for LCA endopeptidase activity against the peptide substrate in the presence and absence of different concentrations of Compound 1. The initial rates kinetics were determined by incubating BoNT/A LC (50 nM) at various concentration of the peptide substrates ([s]=5, 10, 20, 25 μM) and ([I]=0 μM (red, solid), 6.25 μM (purple, long dash), 12.5 μM (green, dash) and 25 μM (blue, round dot). The Lineweaver-Burk plot is best described as intersecting at a common point in between x and y axis, characteristic of a mix inhibition model such that KM (negative reciprocal of x-intercept) is increasing and maximum velocity is decreasing. The values of the kinetic constants K_{M} and Vmax are summarized in Table 1. Each data point represents the mean with the error bar of the three independent assays.

Compound 1 showed a mixed (non-competitive) type of inhibition, where, is Ki<Ki′. The Ki and Ki′ were estimated to be 5.8±0.7 μM and 11.5 ±2.9 μM, respectively. Because Ki≠Ki′, Compound 1 binds both to BoNT/A LC and to BoNT/A LC-SNAP-25 complex with different affinities. Moreover, the data showed both Ki<Ki′ and an increase in Km values (K_{Mapp}, 19.4, 20.5, 21.9 μM) with an increase in the concentration of Compound 1 (3, 6, and 12 μM, respectively) (Table 1), thus indicating that Compound 1 favors binding to free BoNT/A LC more than to the BoNT/A LC-SNAP-25 (enzyme-substrate or ES) complex.

TABLE 1
Kinetic constants (average and standard deviation) of the BoNT/A
LC catalyzed reaction in the presence and absence of Compound 1.
Increasing Michaelis constant (K_{M}) and maximum reaction rate (Vmax)
were observed for Compound 1 are consistent with the mixed type
of inhibition. The K_{M} and Vmax values were determined by non-linear
regression method. [c], Compound 1 concentration.

| Compound 1 [c] | Vₘₐₓ (RFU/s) | K_{M} (μM) |
|---|---|---|
| 0 μM | 3.44 ± 0.1 | 18.37 ± 1.8 |
| 3 μM | 2.13 ± 0.5 | 19.41 ± 2.05 |
| 6 μM | 2.09 ± 0.2 | 20.45 ± 0.32 |
| 12 μM | 1.56 ± 1.1 | 21.91 ± 3.61 |

* Data represent the averages from three independent experiments (mean ± SD, n = 3).

### Effect of Compound 1 on the Intrinsic Fluorescence of BoNT/A LC

The fluorescence measurements of BoNT/A LC (2 μM) at different concentrations of Compound 1 (20 to 200 μM) were measured at two different temperatures (298.15 K and 310.15 K) using ISS K2 Fluorimeter (Champaign, Ill., USA). Protein solutions (0.1 mg/ml) were excited at 295 nm. Emission spectra were recorded between 310 and 400 nm.

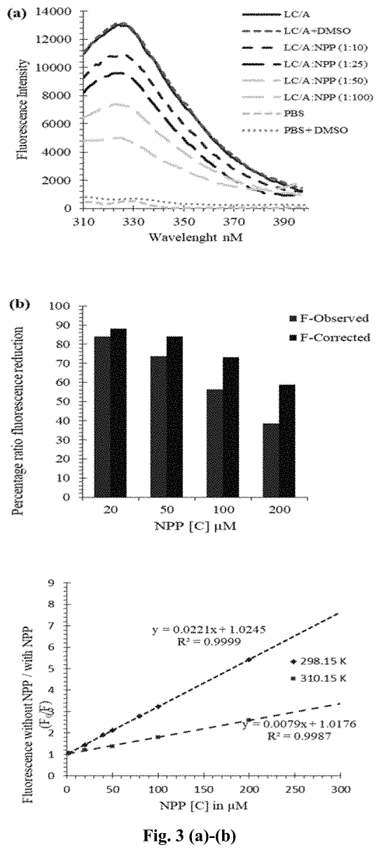

The conformational changes in BoNT/A LC upon binding with Compound 1 were further examined at the tertiary structure level by monitoring intrinsic tryptophan fluorescence in the presence and absence of Compound 1. Different concentrations of Compound 1 were incubated with 2 μM of BoNT/A LC. Since Compound 1 was prepared in DMSO, BoNT/A LC incubated with DMSO was used as a control. FIG. 3a shows a similar fluorescence intensity of BoNT/A LC in the absence (red line) and in the presence (green dotted line) of DMSO. The intrinsic fluorescence of BoNT/A LC remains unchanged in the presence of DMSO (FIG. 3a). Since 280 nm is the wavelength at which tryptophan and tyrosine residues absorb maximally, the excitation at 295 nm was chosen to excite Trp residues, avoiding contribution from Tyr fluorescence, selectively. Emission λmax was observed at 324 nm (FIG. 3a), indicating that Trp residues in BoNT/A LC are buried and constrained in a hydrophobic environment (26, 27). The emission λmax upon excitation at 295 nm for BoNT/A LC treated with Compound 1 was observed at 324 nm (FIG. 3a).

Additionally, quenching of Trp fluorescence of BoNT/A LC was dependent on Compound 1 concentration. The fluorescence intensity at emission maxima shows a drop in intrinsic fluorescence intensity of BoNT/A LC (2 μM) corresponding concentration-dependent addition of NPP (20 μM, 50 μM, 100 μM, and 200 μM) in the ratio of (1:10, 1:25, 1:50, 1:100). Notably, when BoNT/A LC was incubated with DMSO, no reduction of the signal was observed, suggesting that the resulted signal quenching is due to the presence of Compound 1. Compound 1 has a conjugated double bond system, which results in absorption in the region of the excitation and emission wavelengths of BoNT/A LC. These result in the inner filter effect that can be nullify by correcting the emission intensity at λmax of BoNT/A LC (FIG. 3b). Percentage ratio fluoresces signal reduction of F-observed was compared with F-correction when incubated with Compound 1 in the concentration manner. The corrected intensity of Trp fluorescence signal at λmax of BoNT/A LC in the presence of 20, 50, 100 and 200 μM concentrations of Compound 1 still showed a reduction of the signal to 88, 84, 73, and 59%, respectively (FIG. 3b), suggesting Compound 1 might form a complex with BoNT/A LC, hence contributed in the quenching of intrinsic fluorescence of BoNT/A LC.

The Stern-Volmer plots (FIG. 3c) of the fluorescence intensity of BoNT/A LC without Compound 1/with Compound 1 (F0/F) vs different concentrations of Compound 1 at 298 K and 310 K temperature was linear. The Ksv at 25° C. and 37° C. were 22.10×103 M−1 and 7.90×10³ M⁻¹, respectively (Table 2). Both the above observations suggested that the quenching of BoNT/A LC fluorescence by Compound 1 is a static quenching.

TABLE 2
The quenching constants (Kₛᵥ), binding constants (Kₐ) and number of binding sites
(n) and a relative thermodynamic parameter of Compound 1-BoNT/A interaction.

| | Kₛᵥ | | Kₐ | | | ΔH | ΔS | ΔG |
|---|---|---|---|---|---|---|---|---|
| T (K) | (×10³ M⁻¹) | R^{2a} | (×10³ M⁻¹) | n | R^{2b} | (kJ · mol⁻¹) | (kJ · mol⁻¹ K⁻¹) | (kJ · mol⁻¹) |
| 298 | 22.10 | 0.98 | 26.3 | 0.90 | 0.99 | −70.8 | −0.15 | −25.2 |
| 310 | 7.90 | 0.99 | 8.7 | 0.90 | 0.99 | −70.8 | −0.15 | −23.4 |

R^{2a} and R^{2b} is the regression coefficients for Kₛᵥ and Kₐ values, respectively.

Purified BoNT/A LC was subjected to a final gel filtration step using Bio-Rad mini spin size exclusion spin columns (Bio-Rad, California, Mass.) to ensure a complete buffer exchange, as well as to exclude trace amounts of auto cleavage products. All experiments were carried out in the same buffer to control for heat of dilution effects, i.e. 10 mM HEPES (pH 7.4) supplemented with 150 mM NaCl and surfactant 0.5% p-20. The protein concentrations were confirmed by UV-visible absorbance measurements. calorimetric titration was performed three times on a TA instruments-ITC calorimeter (NANO ITC from TA Instruments, New Castle, Del., USA) at 298 K. BoNT/A LC was used at a concentration of 40 μM in the cell, and Compound 1 at a concentration of 400 μM in the injection syringe. Prior to the titration, the samples were degassed for 10 min. The positive deflections observed at the end of the titration reflected the enthalpy of dilution of the Compound 1 solution and were subtracted from the binding data. The analysis of the data was done with Nano Analyzer Software 2.2.4 (TA Instruments) using independent sites model setup to obtain the following parameters: a number of binding sites (N), binding enthalpy (ΔH), and binding constant (KD).

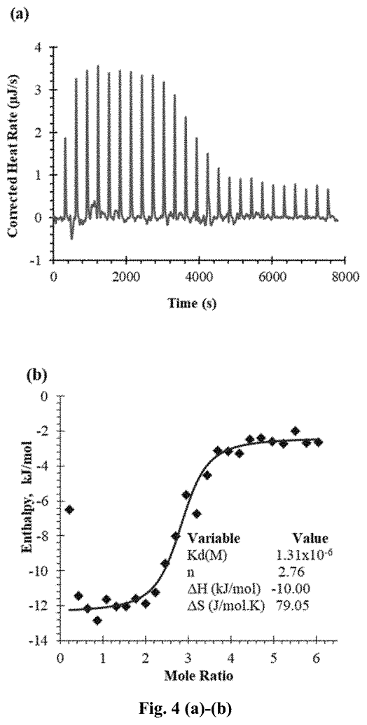

Compound 1 interacts with the BoNT/A LC using ITC. The experiments were carried out to determine the stoichiometry, entropy, enthalpy, and association constant (Table 3). The binding stoichiometry (n) of Compound 1 to BoNT/A LC is 2.8 (FIG. 4b). This value indicates that ligand binds on 2-3 sites on the enzyme surface, which means that BoNT/A possesses multiple binding sites for Compound 1.

TABLE 3
Thermodynamic parameter of Compound 1 binding with BoNT/A
LC: temperature (T), association constant (Kₐ), change in enthalpy
(ΔH), dissociation constant (K_{d}), change in entropy (ΔS),
Gibbs free energy (ΔG) and a number of binding sites (N)
obtained from nano analyze software, TA instruments.

| Parameter | Value |
|---|---|
| −T (° C.) | 25 |
| Kₐ (1/M) | 7.609 × 10⁵ |
| ΔH (kJ/mol) | −10.00 |
| Kd (M) | 1.314 × 10⁻⁶ |
| ΔS (J/mol · K) | 79.05 |
| ΔG (kJ/mol) | −33.55 |
| N | 2.760 |

We Claim:

1. A method of treating an individual suffering from botulism comprising:
administering to the individual a composition comprising a therapeutically effective amount of Compound 1 or its pharmaceutically acceptable salts or derivatives thereof; wherein the Compound 1 is

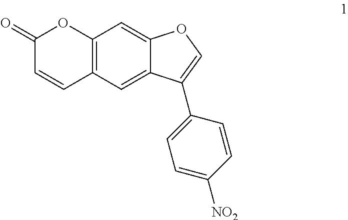 [SMILES: O=c1ccc2cc3c(-c4ccc([N+](=O)[O-])cc4)coc3cc2o1]

2. The method of claim 1, wherein the botulism is caused by botulinum neurotoxin produced by Clostridium botulinum.

3. The method of claim 1, wherein Compound 1 inhibits botulinum neurotoxin.

4. The method of claim 3, wherein the botulinum neurotoxin is type A, B, C, D, E, F, or G.

5. The method of claim 1, wherein the therapeutically effective amount is in the range of <1 to 10 μM/Kg.

6. A composition comprising a therapeutically effective amount of 3-(4-nitrophenyl)-7H-furo[3,2-g]chromen-7-one or its pharmaceutically acceptable salts and one or more pharmaceutically acceptable excipients.

7. The composition of claim 6, wherein the pharmaceutically acceptable excipient is selected from the group consisting of diluents, carriers or fillers, binders, disintegrants, lubricants, suspending agents, solubilizing agents/surfactants, stabilizing agents, glidants, antioxidants, colors, flavors, preservatives, or mixtures thereof.

8. The composition of claim 7, wherein the diluent is selected from the group consisting of ethanol, glycerol, Dimethyl sulfoxide (DMSO), water, or mixture thereof.

9. The composition of claim 7, wherein the carrier is selected from a group consisting of lactose, starch, glucose, methylcellulose, magnesium stearate, mannitol, sorbitol, or mixtures thereof.

10. The composition of claim 7, wherein the binder is selected from the group consisting of starch, gelatin, natural sugars such as glucose, anhydrous lactose, free-flow lactose, beta-lactose, corn sweeteners, natural and synthetic gums, or mixtures thereof.

11. The composition of claim 10, wherein the natural and synthetic gum is selected from the group consisting of acacia, tragacanth or sodium alginate, carboxymethyl cellulose, and polyethylene glycol, or mixtures thereof.

12. The composition of claim 7, wherein the lubricant is selected from the group consisting of sodium oleate, sodium stearate, magnesium stearate, sodium benzoate, sodium acetate, sodium chloride, or mixtures thereof.

13. The composition of claim 6, wherein the pharmaceutically acceptable excipient is selected from the group consisting of aspartame, phenylalanine, benzalkonium chloride, benzoic acid and benzoates, benzyl alcohol, boric acid and borate, cyclodextrins, dextrans, ethanol, fructose, sorbitol, lactose, phosphates, polysorbates, proline, propylene glycol and esters, sodium lauryl sulfate, wheat starch (containing gluten), gelatin, cellulose, cellulose derivatives, polyvinylpyrrolidone, starch, or mixtures thereof.

14. The composition of claim 6, wherein the composition further comprises one or more lipids wherein the one or more lipid is a charged lipid or a mixture of charged and neutral lipids.

15. The composition of claim 14, wherein the one or more lipids is N-glutarylphosphatidylethanolamines (Glutaryl PE)

16. The composition of claim 15, further comprises a polyethylene sorbitol ester (Tween 80).

17. The composition of claim 14, wherein the one or more lipids are composed of oily solutions selected from the group consisting of triglyceride, ethyl icosapentate, tocopherol nicotinate, teprenone, indomethacin franesil, and dronabinol.

18. The composition of claim 6, wherein Compound 1 is encapsulated in microspheres, liposomes, or nanoparticles linked to detoxified recombinant BoNT.

19. The method of claim 1, wherein the composition is administered by oral administration, nasal administration, topical administration, parenteral administration, rectal administration, systemic administration, intramuscular administration, or intravenous administration.

20. The method of claim 1, wherein the method comprises administering one or more other drugs in combination with Compound 1.
